(19) United States
(12) Patent Application Publication
(10) Pub. No.: US 2014/0120324 A1
(43) Pub. Date: May 1, 2014

(54) IMPLANTABLE DEVICES WITH CORRODIBLE MATERIALS AND METHOD OF MAKING SAME

(71) Applicant: W. L. Gore & Associates, Inc., Newark, DE (US)

(72) Inventor: Edward H. Cully, Flagstaff, AZ (US)

(73) Assignee: W. L. Gore & Associates, Inc., Newark, DE (US)

(21) Appl. No.: 14/066,338

(22) Filed: Oct. 29, 2013

Related U.S. Application Data
(60) Provisional application No. 61/720,110, filed on Oct. 30, 2012.

(51) Int. Cl. A61L 31/02; A61F 2/07; A61L 31/04; B29C 65/00

(52) U.S. Cl. CPC A61L 31/022; B29C 65/002; A61F 2/07; A61L 31/048
USPC 428/188; 156/155; 623/1.15

(57) ABSTRACT

The present disclosure describes implantable medical devices comprising at least one channel comprising a plurality of particles capable of undergoing a chemical reaction resulting in an increase in the rigidity of the channel. The medical device can be a stent, stent graft, occlusion bag, or other device configured.

### FIELD

The present disclosure relates generally to implantable devices and, more specifically, to implantable medical devices comprising channels encasing a material capable of volumetrically increasing or capable of forming a more rigid construct through chemical interactions of the components of which the material is comprised before and/or after implantation within the body of a patient.

### BACKGROUND

Implantable medical devices are frequently used to treat the anatomy of patients. Such devices can implanted in the anatomy to provide treatment to the patient. Many devices provide support to the treatment area, such as, for example, by supporting the walls of a blood vessel.

Accordingly, there is a need for medical devices that provide sufficient support to treatment areas of patients and can be easily and efficiently manufactured.

### BRIEF DESCRIPTION OF THE DRAWINGS

The accompanying drawings are included to provide a further understanding of the disclosure and are incorporated in and constitute a part of this specification, illustrate embodiments of the disclosure, and together with the description, serve to explain the principles of the disclosure, wherein;

FIGS. 1A and 1B illustrate a side view and a cross sectional view, respectively, of a medical device in accordance with the present disclosure;

FIG. 2 illustrates a side view of a medical device in accordance with the present disclosure;

FIGS. 3A and 3B illustrate a side view and a cross sectional view, respectively, of a medical device in accordance with the present disclosure; and

FIGS. 4A and 4B illustrate cross sectional views of medical devices in accordance with the present disclosure.

### DETAILED DESCRIPTION OF THE ILLUSTRATED EMBODIMENTS

Persons skilled in the art will readily appreciate that various aspects of the present disclosure can be realized by any number of methods and systems configured to perform the intended functions. Stated differently, other methods and systems can be incorporated herein to perform the intended functions. It should also be noted that the accompanying drawing figures referred to herein are not all drawn to scale, but can be exaggerated to illustrate various aspects of the present disclosure, and in that regard, the drawing figures should not be construed as limiting.

As used herein, “medical devices” can include, for example, stents, grafts, stent-grafts, filters, valves, occluders (such as aneurysm occluding bags), markers, sensors, prostheses, bandages, snares, constrictors, therapeutic agent delivery devices, devices for repairing arteriovenous malformations and trauma damage, and other endoluminal and implantable devices that are implanted, temporarily or chronically, in the vasculature or other body conduit or cavity at a treatment site.

The medical devices and medical device components described herein can be corrodible. As used herein, “corrodible” means capable of undergoing a chemical reaction that causes a change in the chemical composition, such as through oxidation or reduction. Corrodible can also mean biocorrodible, biodegradable, bioabsorbable, bioresorbable, bioerodible, or otherwise capable of being dissolved and/or deteriorated within the body of a patient.

Embodiments of the present disclosure are directed toward constructs, which comprise a channel or casing wherein the channel can be filled or inflated with a plurality of corrodible particles. Said particles can be introduced into the channel in the form of dry particles or a suspension, i.e., a carrier fluid plus particles. Said particles can undergo a corrosion reaction, resulting in increased rigidity of the channel. Said particles can volumetrically expand upon undergoing a chemical reaction with reagents present in a carrier fluid, casing material, and/or surrounding environment in general. Said particles can comprise chemical elements and/or compounds from which corrosion products are formed and bond to each other or other chemical species also present in the carrier fluid, casing material, and/or surrounding environment in general, resulting in a more rigid channel or casing. Said channel or casing can be embedded within or overlaid on a supporting surface, e.g., a graft member. Said channel or casing can form a supporting framework, which increases the strength of the supporting surface, in a radial, diagonal, transverse, and/or longitudinal direction. In various embodiments, said casing or channel comprising said particles can form a stent or stent-graft.

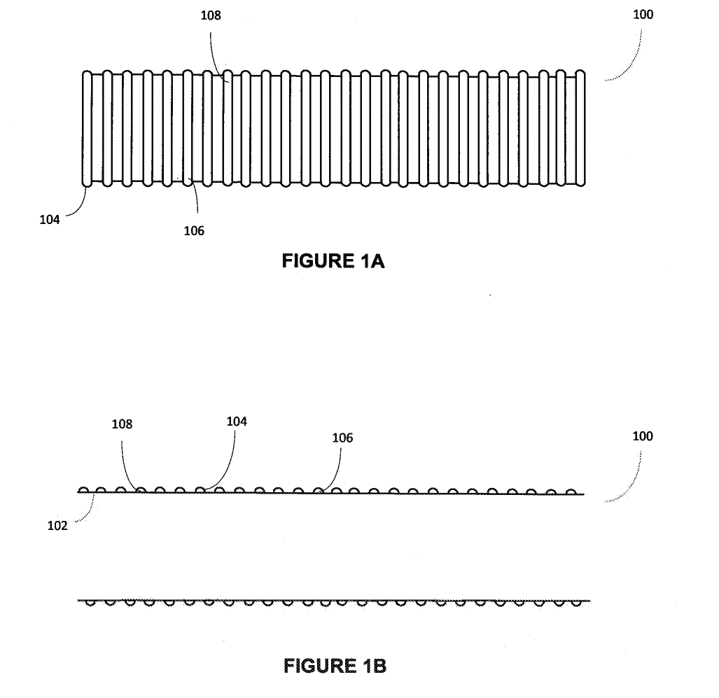

For example, with reference to FIGS. 1A and 1B, a medical device 100 can comprise a first graft member 102 and a second graft member 104 between which a channel 106 is defined and filled with a plurality of corrodible particles 108. The corrodible particles 108 can comprise chemical elements and/or compounds that undergo corrosion reactions, the corrosion products of which result in increased rigidity of the channel. As such, the channel or network of channels 106 forms a supporting framework. In various embodiments, both first graft member 102 and second graft member 104 can be configured to provide a circulatory bypass route to avoid vessel damage or abnormalities, such as aneurysms and/or to provide a structure that can aid in remodeling a vessel. In an embodiment, medical device 100 can be collapsed to a delivery profile, mounted on a delivery catheter, and balloon expanded once at the delivery site.

The plurality of particles 108 can be injected into channel 106 in the form of dry particles or dry mixture of particles and reagents, or in the form of a suspension. In various embodiment, the suspension comprises a carrier fluid and a plurality of particles 108. In other embodiments, dry particles are introduced first and then a reagent solution can be introduced/injected at a later time, such as in vivo. In various embodiments, channel 106, e.g., graft members 102 and/or 104, can comprise a material that is permeable to a carrier fluid or reagent solution so that the bulk of a fluid can be drained, evaporated, or otherwise extracted from channel 106. In an embodiment, only one portion of channel 106 or graft member 102, 104 is permeable so that a fluid exits in a preferential direction, e.g. on a luminal side, and the other portion of channel 106 or the other graft member 102, 104 is substantially impermeable to a fluid in a preferential direction, e.g., on an abluminal side.

The carrier fluid or reagent solution can be any suitable liquid, which is biocompatible, such as saline solutions, water, ethanol, propylene glycol, or the like. In addition, the carrier fluid or reagent solution can further comprise reagents to facilitate a corrosion reaction of the corrodible particles 108. For example, in an embodiment, a suspension or reagent solution can comprise an aqueous solution saturated with an oxidizing agent, such as oxygen. In a further embodiment, a suspension or reagent solution can contain corrosion enhancers, such as a salt, that facilitate, initiate, and/or accelerate the corrosion reaction. The carrier fluid or reagent solution solvent itself can be a corrosion enhancer and/or a reagent. Such a solution can facilitate a rapid corrosion reaction of the particles resulting in increased rigidity of the channel due to a volumetric increase and/or bonded corrosion products.

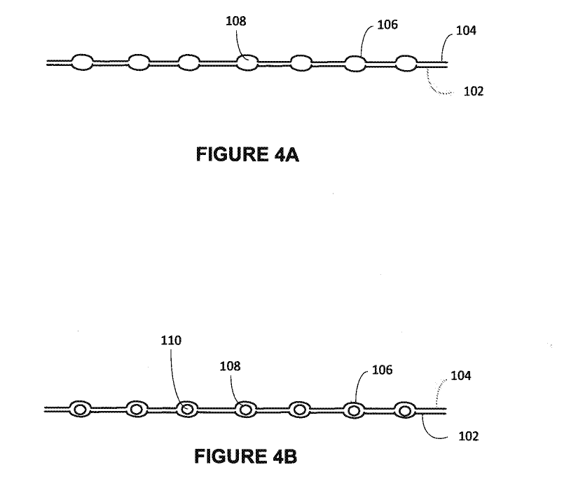

In various embodiments, with reference to FIG. 4A, channel 106 is any structural feature configured to encase corrodible particles 108, e.g., between a surface of first graft member 102 and a surface of second graft member 104. For example, in an embodiment, medical device 100 can comprise a single channel 106 that is disposed along at least a portion of the overlapping segment of second graft member 104 and first graft member 102.

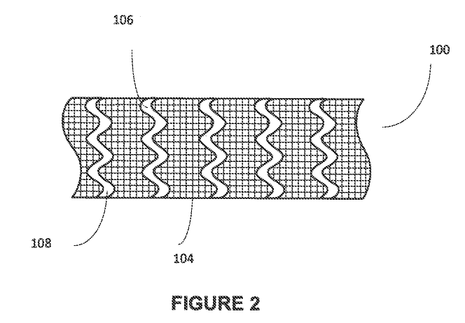

With reference to FIG. 2, medical device 100 can comprise any number or pattern of channels 106 that facilitate a supporting framework. In various embodiments, medical device 100 can comprise a plurality of channels 106 and/or a network of channels 106. Channel 106 can be oriented longitudinally, diagonally, helically, circumferentially, or combinations thereof. Channels can be curved, straight, undulating, and/or combinations thereof. Channels can form a regularly repeating pattern, be randomly/chaotically arranged, or combinations thereof. Channels 106 can be parallel, perpendicular, or askew. Channel 106 can be in fluid communication with at least one other channel 106, forming a network, or isolated from one another. In various embodiments, channel 106 can comprise a pattern that is similar to the framework of a stent. A medical device comprising any configuration of channels is within the scope of the present disclosure.

Channels 106 can be formed in medical device 100 by a number of different techniques. For example, with reference to FIG. 4A, channels 106 can be created by selectively applying an adhesive to (e.g., fluorinated ethylene propylene) and/or local thermal bonding (which can be facilitated by a laser-induced heat treatment) of one or more surfaces of first graft member 102 and second graft member 104 in a manner that defines channel 106. In various embodiments, the exterior surface of a tubular first graft member 102 is in contact with an interior surface of a tubular second graft member 104. One or more channels 106 can be formed by coupling exterior surface of first graft member 102 and interior surface of second graft member 104, at least along channel 106 boundaries. In such embodiments, graft members 104, 106 are coupled along a pattern of lines that define a desired pattern of channel 106. In other embodiments, graft members are coupled along an entire surface except the portion of the surface that channel 106 will occupy.

In yet other embodiments, with reference to FIG. 4B, channel 110 can comprise a elongated structure having a sealed lumen and can extend along and be coupled to a supporting substrate 102 (either an inner or outer surface) or alternatively, extend between two supporting substrates 102, 104. For example, channel 110 can comprise a tube coupled to a supporting substrate 102, 104. As illustrated in FIG. 4B, in various embodiments, channel 110 is positioned between the exterior surface of tubular first graft member 102 and the interior surface of tubular second graft member 104. Similarly, channel 110 can be configured in any number and pattern as discussed above in connection with channels 106.

As previously described, channels of medical device are configured to encase corrodible particles. For example, with reference to FIG. 1A, channels 106 disposed in medical device 100 contain corrodible particles 108. For example, corrodible particles 108 can be injected into channels 106 after formation of graft 102, 104 and channel 106 construct. In an embodiment, the site of injection is then sealed to prevent the release of particles 107. However, any method of supplying corrodible particles 108 to channels 106 of medical device 100 is within the scope of the present disclosure.

Corrodible particles 108 can be any type, size, mixture, or shape of particle that can flow and fill channel 106. Particles 108 can be fine grained, e.g., a powder, grit, or the like. The grain size and shape of particles 108 can be adjusted to adjust reaction kinetics.

In various embodiments, corrodible particles 108 can be configured to increase in volume upon reacting with a reagent. Stated differently, the reaction products occupy a greater volume than particles 108. For example, corrodible particles 108 can increase in volume once injected into channel 106 with other reactants or corrosion enhancers present in the suspension, graft material, and/or surrounding environment in general. In other embodiments, corrodible particles 108 can increase in volume once placed in the body and undergoing a slow reaction process upon exposure to biological fluids in vivo. With reference to FIG. 1A, medical device 100 comprises a graft 102,104 having a plurality of channels 106 configured to accept corrodible particles 108. After the graft is deployed, and corrodible particles are introduced or activated, the volume of corrodible particles 108 increases, thereby increasing the pressure exerted against the walls of channel 106. This increased pressure can create increased support and rigidity provided by medical device 100. In various embodiments, corrodible particles 108 allows medical device 100 to operate as a stent-graft, providing support to a treatment area, such as, for example, the vasculature, of a patient.

In various embodiments, after placement in channels 106, corrodible particles 108 can undergo a chemical reaction that causes the reaction products to form a chemical bond with other reaction products, other corrodible particles, the graft member 102, 104, or other reagents in the suspension. For example, corrodible particles 108 can comprise reaction products that form cations and/or anions, which can react with other cations and/or anions, to form an ionic bond, such ferrous hydroxide forming upon oxidation of iron. As these particles corrode, the number of ionically bonded elements and/or compounds increases, increasing the rigidity of the channel.

In various embodiments, corrodible particles 108 can be metallic and can include suitable biocompatible metal or metal alloy, such as iron, magnesium, zinc, tungsten, alloys of thereof, or combinations thereof In addition, corrodible particles 108 can comprise particles of other metals and/or metal alloys that can corrode or oxidize within the body without producing harmful or deleterious byproducts. However, corrodible particles 108 can comprise any chemical constituent capable of increasing in volume or forming chemically bonded agglomerations in a safe and non-harmful way within the body.

In an embodiment, corrodible particles 108 can comprise an iron alloy. An iron alloy refers to a metal composition with iron (Fe) present as the major component. In various embodiments, an iron alloy can comprise at least 50% iron. In various embodiments, an iron alloy can comprise at least 60% iron. In various embodiments, an iron alloy can comprise at least 70% iron. In various embodiments, an iron alloy can comprise at least 80% iron. In various embodiments, an iron alloy can comprise at least about 90% iron. In various embodiments, an iron alloy can comprise at least about 95% iron. In addition to the elemental iron, iron alloys for use in bio-corrodible medical devices can comprise non-iron elements such as carbon, nickel, cobalt, manganese, magnesium, lithium, calcium, chromium, molybdenum, tantalum, platinum, palladium, vanadium, iridium, rhenium, rhodium, rubidium, osmium, tungsten, titanium, niobium, zirconium, hafnium, aluminum, boron, sulfur, tin, silicon, yttrium, zinc, bismuth, silver, copper, iridium, indium, tin, and any lanthanide or actinide. In various embodiments, an iron alloy for a medical device designed to corrode in the body can comprise from 0 to about 40% manganese, 0 to about 5% chromium, 0 to about 10% nickel, 0 to about 25% cobalt, 0 to about 1% aluminum, 0 to about 5% molybdenum, 0 to about 3% titanium, 0 to about 3% zirconium, 0 to about 1% carbon, 0 to about 1% silicon, 0 to about 3% niobium, 0 to about 1% nitrogen, and 0 to about 1% yttrium, with the remainder iron. Examples of iron alloys for use in bio-corrodible medical devices in accordance with the present disclosure can be found in U.S. Pat. No. 8,246,762 to Janko et al., the content of which is incorporated herein by reference in its entirety.

In various embodiments, both first graft member 102 and second graft member 104 can comprise a polymeric membrane. The graft can be biodegradable polymer and/or promote tissue in growth. Polymeric grafts include expanded polytetrafluoroethylene (ePTFE), polyester, polyurethane, fluoropolymers, such as perfluoroelastomers and the like, polytetrafluoroethylene, silicones, urethanes, ultra high molecular weight polyethylene, polyethylenes such as Dacron®, aramid fibers, and combinations thereof. Other embodiments for a graft member material can include high strength polymer fibers such as ultra-high molecular weight polyethylene fibers (e.g., Spectra®, Dyneema Purity®, etc.) or aramid fibers (e.g., Technora®, etc.). In addition to the materials enumerated above, any material that is biocompatible and suitable for providing a supporting surface for the channel is within the scope of the present disclosure. The graft member can be treated, coated, or imbibed with a beneficial agent. In various embodiments, the porosity of the first or second 102, 104 graft member or at least a portion of the elongated structure defining a sealed lumen can be engineered so as to facilitate permeation of the carrier fluid yet “filter” and entrap the corrodible particles. The corrodible particles will densely pack into the space provided and begin the oxidation process, which in turn will solidify the particles into a structure.

In various embodiments, a surface of graft member 102, 104 can comprise a layer that allows for tissue in growth. Facilitating in growth permits graft member 102, 104 to be incorporated into the vessel wall after a period of time. In an embodiment, the surface of graft member 102,104 can comprise a material that has an open, porous microstructure. An open microstructure provides an uneven surface topography having crevices, tunnels, and cavernous features within which cells and tissue(s) can occupy. Similarly, in other embodiment, the surface can comprise an open macrostructure that can facilitate tissue in growth, e.g., a surface providing ridges that provide an uneven surface topography providing surface features within which cells and tissue(s) can occupy. In addition, the outer surface can be coated or treated with beneficial agents that enhance the rate of tissue in growth. For example, in an embodiment, a beneficial agent can comprise a pro-angeogenic agent, such as a vascular endothelial growth factor.

In various embodiments, at least a portion of first graft member 102 is concentrically surrounded by second graft member 104. For example, as illustrated in FIGS. 1A and 1B, first graft member 102 and second graft member 104 can be generally tubular members that each defines a lumen. In other embodiments, first graft member 102 and second graft member 104 can be substantially circular members. For example, first and second graft members 102 and 104 can form a generally circular bag for use in occluding an aneurysm. However, any configuration in which second graft member 104 concentrically at least a portion of first graft member 102 is within the scope of the present disclosure.

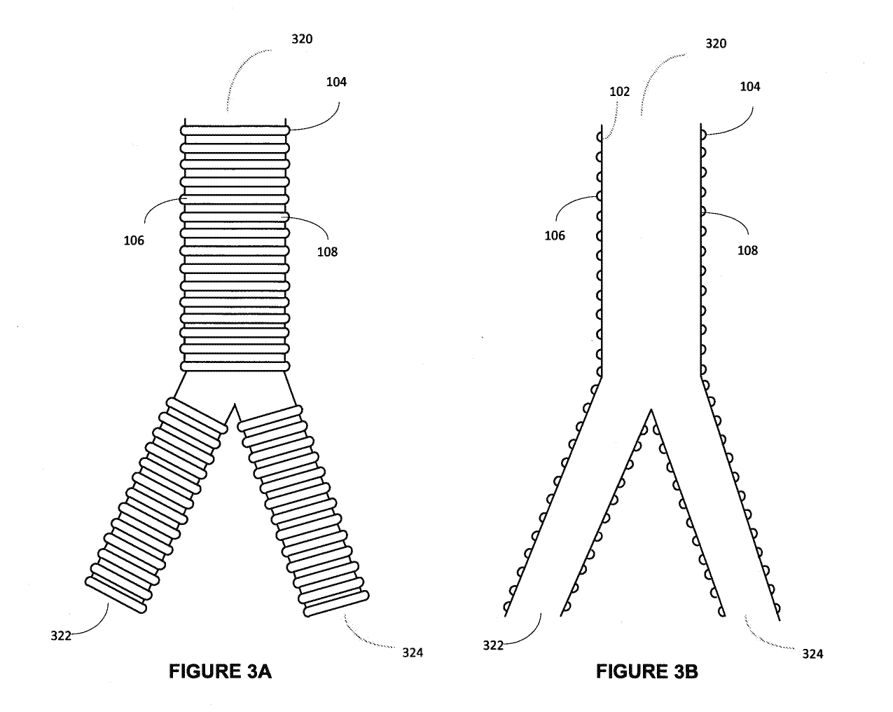

As illustrated in FIGS. 3A and 3B, medical device 100 can comprise, among other configurations, first graft member 102 and second graft member 104, wherein both graft members are in substantially bifurcated configurations. For example, medical device 100 comprises first and second graft members 102 and 104 in generally bifurcated configurations, such that a main lumen 320 and two branched lumens 322 and 324 are formed. Medical device 100 further comprises a plurality of channels 106 that surround and are generally perpendicular to main lumen 320 and branched lumens 322 and 324.

Further, medical devices in accordance with the present disclosure can comprise configurations different from those previously discussed. For example, a medical device can comprise a polymeric sheet shaped as a balloon and/or bag. The resulting balloon or bag can be filled with a plurality of particles and/or suspension. Said suspension can comprise metallic particles, a carrier fluid such as water, a corrosion enhancer such as a salt, and a reagent such as oxygen. This balloon or bag can be used to occlude aneurysms by placing the bag within an aneurysm and injecting the particles into the inner space, causing the volume of the bag to increase, thereby filling and occluding at least a portion of the aneurysm. However, any configuration of medical device that requires a space to be filled with a plurality of particles which are subsequently solidified or otherwise increase the rigidity of the medical device is within the scope of the present disclosure.

In accordance with another aspect of the invention, a method of making can comprise providing a graft member having a channel, or a pattern thereof. The graft member having a channel can be constructed by adhering two film members in a manner that outlines a channel. Such graft members can be in tubular form. Once formed, the channels can be supplied with a plurality of particles in a dry, inactive state. This device embodiment may then be mounted upon its delivery system. In this fashion, the corrosion process can be controlled. For example, a reagent, e.g., oxygen and/or water, can be introduced into the channel at a desired time. In an embodiment, reagents can be introduced in vivo, through injection or permeation, e.g., as or with physiological fluids.

Once initiated, a chemical reaction causes an increase in volume and/or a chemical bond between the reaction products and other reactions products or compounds or elements in the graft member and/or the suspension.

Once a described device is constructed, the device can be collapsed onto a catheter about an expandable member such as a balloon. The delivery system will provide 1) for a method of distension (such as underlying balloon) and 2) for a means for delivering an activation fluid to the space containing the corrodible particles.

In accordance with another embodiment, a method of delivery can comprise providing a described graft member having empty channels. In other words, the device is fabricated without indwelling corrodible particles. The device is mounted upon its delivery system which, 1) provides for a method of distension (such as underlying balloon) and 2) provides for mechanism of delivering the suspension (containing the corrodible particles) to the channel(s). Placing the device at the site, deploying the described device and finally injecting the suspension into the channels(s) where they will begin an oxidation process resulting in solidifying into a support structure. In an embodiment, the graft can be self-sealing to facilitate the channel sealing after the device, such as a cannulae or tube, that injects the suspension is removed from the channel.

It will be apparent to those skilled in the art that various modifications and variations can be made in the present disclosure without departing from the spirit or scope of the disclosure. Thus, it is intended that the present disclosure cover the modifications and variations of this disclosure provided they come within the scope of the appended claims and their equivalents.

Likewise, numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications can be made, especially in matters of structure, materials, elements, components, shape, size and arrangement of parts including combinations within the principles of the disclosure, to the full extent indicated by the broad, general meaning of the terms in which the appended claims are expressed. To the extent that these various modifications do not depart from the spirit and scope of the appended claims, they are intended to be encompassed therein.

What is claimed is:

1. An implantable medical device comprising:
a polymeric sheet having at least one channel; and
a plurality of corrodible particles located in the at least one channel.

2. The device of claim 1 wherein the plurality of particles are at least one of an iron alloy and a magnesium alloy.

3. The device of claim 1, wherein the plurality of particles are at least partially oxidized.

4. The device of claim 3, wherein the plurality of particles is a metallic powder.

5. The device of claim 4, wherein a volume of the corroded particles is greater than a volume of the plurality of particles prior to corrosion.

6. The device of claim 1 wherein the polymeric sheet comprises an expanded polytetrafluoroethylene.

7. The device of claim 1 wherein the polymeric sheet comprises a bioabsorbable polymer.

8. The device of claim 1, wherein the at least a portion of a channel comprises a permeable material.

9. The device of claim 1, wherein the polymeric sheet is configured as a tubular member that defines a lumen extending there through.

10. The device of claim 1, wherein the at least one channel defines a stent pattern.

11. A method of making a polymeric graft comprising:
providing a first tubular form and a second tubular form;
disposing the first tubular form around the second tubular form;
forming at least one channel between the first tubular form and the second tubular form; and
placing into the channel a plurality of corrodible particles.

12. The method of claim 11, wherein the at least one channel is formed by adhering discrete sections of the first tubular form to discrete sections of the second tubular form in a desired pattern of the at least one channel.

13. The method of claim 11, wherein the at least one channel is formed by laser treatment of the first tubular form and the second tubular form in a desired pattern of the at least one channel.

14. The method of claim 11, wherein the at least one channel is formed by placing a channel tube between an interior surface of the second tubular form and an exterior surface of the first tubular form.

15. The method of claim 11, wherein the step of filling the channel with a suspension comprising metallic particles comprises injecting the suspension into the channel.

16. The method of claim 11, wherein the at least a portion of the channel is water permeable.

17. The method of claim 11, wherein the metallic particles are corrodible.

18. The method of claim 11, further comprising initiating corrosion of the metallic particles to form corrosion products.

19. The method of claim 18, wherein the corrosion products have a volume and the volume of the corrosion products is greater than the volume of the metallic particles prior to corrosion of the metallic particles.

20. The method of claim 18, wherein the corrosion products form a chemical bond with other corrosion products.

21. The method of claim 11, wherein the at least one channel defines a stent pattern.
